(19) United States
(12) Patent Application Publication
(10) Pub. No.: US 2023/0181143 A1
(43) Pub. Date: Jun. 15, 2023

(54) RADIOGRAPHIC CONTROL APPARATUS, RADIOGRAPHIC SYSTEM, RADIOGRAPHIC CONTROL METHOD, AND STORAGE MEDIUM

(71) Applicant: CANON KABUSHIKI KAISHA, Tokyo (JP)

(72) Inventor: YASUTOMO SHIMIZU, Tokyo (JP)

(21) Appl. No.: 18/063,025

(22) Filed: Dec. 7, 2022

(30) Foreign Application Priority Data
Dec. 15, 2021 (JP) 2021-203549

(51) Int. Cl. A61B 6/00; A61B 5/00; G06T 7/593

(52) U.S. Cl. CPC A61B 6/547; A61B 6/4028; A61B 5/0077; A61B 6/464; A61B 6/467; A61B 6/563; G06T 7/593; G06T 2207/20081; G06T 2207/10124

(57) ABSTRACT

A radiographic control apparatus includes an obtaining unit configured to obtain position information about a transmission unit configured to transmit a radio wave based on a radio field intensity of the transmission unit and position information about a reception unit configured to receive the radio wave, the transmission unit being disposed on a radiographic apparatus configured to generate a radiographic image of a subject based on radiation emitted from a radiation generation apparatus, and a correction unit configured to correct the position information about the transmission unit using depth information about an area to which the radiographic apparatus belongs in an optical image captured by an imaging apparatus, the depth information being obtained from the optical image.

### BACKGROUND

### Field of Disclosure

The present disclosure relates to a radiographic control apparatus, a radiographic system, a radiographic control method, and a computer-readable storage medium.

### Description of Related Art

Imaging systems using radiation have been known as radiographic systems in the medical field. With digitization of the imaging systems using radiation, a system where an object is irradiated with radiation emitted from a radiation generation apparatus, and a radiographic apparatus detects radiation transmitted though the object and generates a digital radiographic image so that the radiographic image can be observed immediately after radiographic imaging has become prevalent. This process improves a workflow and enables imaging in short cycles compared to conventional film-based imaging methods.

In capturing an image in such an imaging system using radiation, the orientation of a patient and the position of the radiographic apparatus are desirably adjusted depending on the imaging condition (such as an imaging site and a distance between an X-ray tube and a detector) set in the imaging system in advance. Specifically, positioning is performed so that the patient comes between the radiation generation apparatus and the radiographic apparatus and the patient's imaging site is included in an area to be irradiated with the radiation (irradiation field). In the cases of carrying the radiographic apparatus outside a radiographic room and inserting the radiographic apparatus between the patient and a bed, as in imaging during the rounds in particular, there has been an issue of the radiographic apparatus being hidden behind the patient and the positioning of the patient becoming difficult.

To address such an issue, some recent imaging systems using radiation have the following configuration.

Japanese Patent Application Laid-Open No. 2019-33826 discusses an imaging system where an optical camera is attached to a radiation generation apparatus and a captured image such as an optical image captured by the optical camera during radiographic imaging is displayed on a screen of the imaging system. In addition, a position detection unit located within the field of view of the optical camera obtains an optical image of the radiographic apparatus sideways. The position of the radiographic apparatus is calculated from a positional relationship between the position detection unit and the optical camera, and calculated position information is superimposed on the screen of the imaging system. This enables the positioning of the patient and the radiographic apparatus without a hitch even in a state where the radiographic apparatus is hidden behind the patient.

Japanese Patent Application Laid-Open No. 2019-33826 also discusses a method for calculating the position of the radiographic apparatus based on radio field intensity using an electromagnetic wave source on the position detection unit and an electromagnetic wave detection sensor on the radiographic apparatus. Japanese Patent Application Laid-Open No. 2009-28426 discusses moving the position of a radiographic apparatus by using a method for calculating the position of the radiographic apparatus based on radio field intensity using an electromagnetic wave source on the radiographic apparatus and an electromagnetic wave detection sensor on a bed.

The related art is predicated on the position detection unit being located within the field of view of the optical camera and the side of the radiographic apparatus being recognizable to the position detection unit in calculating the position of the radiographic apparatus. In an imaging environment where such conditions are not satisfied, the position of the radiographic apparatus is unable to be calculated to superimpose position information on the screen of the imaging system. The related art solves the issue of visibility of the radiographic apparatus by calculating the position of the radiographic apparatus based on the radio field intensity. However, the radio field intensity of electromagnetic waves is known to typically attenuate due to obstacles. In the case where the radiographic apparatus is hidden behind the patient, the human body serves as an obstacle that causes attenuation of the radio waves and makes the exact position of the radiographic apparatus difficult to be calculated.

### SUMMARY OF THE DISCLOSURE

According to an aspect of the present disclosure, a radiographic control apparatus includes a position information obtaining unit configured to obtain position information about a transmission unit configured to transmit a radio wave based on a radio field intensity of the transmission unit and position information about a reception unit configured to receive the radio wave, the transmission unit being disposed on a radiographic apparatus configured to generate a radiographic image of a subject based on radiation emitted from a radiation generation apparatus, and a correction unit configured to correct the position information about the transmission unit using depth information about an area to which the radiographic apparatus belongs in an optical image captured by an imaging apparatus, the depth information being obtained from the optical image.

Further features of the present disclosure will become apparent from the following description of exemplary embodiments with reference to the attached drawings.

### BRIEF DESCRIPTION OF THE DRAWINGS

FIG. 1 is a diagram illustrating an example of a system configuration of a radiographic system according to a first exemplary embodiment.

FIG. 2 is a diagram illustrating an example of a hardware configuration of a radiographic control apparatus in the radiographic system according to the first exemplary embodiment.

FIG. 3 is a diagram illustrating an example of a software configuration of the radiographic control apparatus in the radiographic system according to the first exemplary embodiment.

FIGS. 4A and 4B are diagrams illustrating an example of a sequence for the radiographic system according to the first exemplary embodiment.

FIG. 5 is a flowchart illustrating an example of distance and position information derivation processing by a radio wave transmission apparatus position calculation unit according to the first exemplary embodiment.

FIGS. 6A, 6B and 6C are diagrams illustrating examples of image display by an image display unit according to the first exemplary embodiment.

FIG. 7 is a flowchart illustrating an example of depth information determination processing by an optical image area determination unit according to the first exemplary embodiment.

FIGS. 8A, 8B, and 8C are diagrams illustrating examples of radio wave transmission apparatus area display by the image display unit according to the first exemplary embodiment.

FIG. 9 is a diagram illustrating an example of a system configuration of a radiographic system according to a second exemplary embodiment.

FIG. 10 is a diagram illustrating an example of a software configuration of a radiographic control apparatus in the radiographic system according to the second exemplary embodiment.

FIGS. 11A, 11B, and 11C are a flowchart illustrating an example of distance and position information derivation processing by a radio wave transmission apparatus position calculation unit according to the second exemplary embodiment.

FIG. 12 is a flowchart illustrating an example of depth information determination processing by an optical image area determination unit according to the second exemplary embodiment.

### DESCRIPTION OF THE EMBODIMENTS

One exemplary embodiment is directed to improving the accuracy of the position calculation of a radiographic apparatus.

The present disclosure is not limited to the foregoing improvements, but also directed to providing an operation and advantageous effects that are derived from configurations described in the following embodiments.

Various exemplary embodiments will be described below with reference to the drawings.

Note that the present disclosure is not limited to the specifically disclosed exemplary embodiments, and various modifications and changes may be made without departing from the scope of the claims.

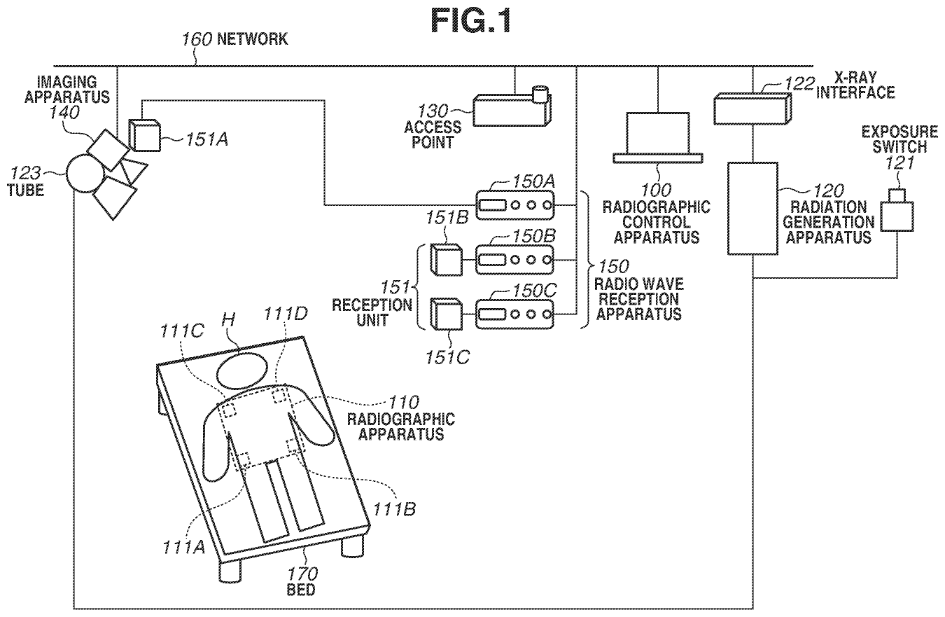

A system configuration according to a first exemplary embodiment will be described with reference to FIGS. 1 to 3.

FIG. 1 illustrates a configuration example of an entire radiographic system according to the present exemplary embodiment. The radiographic system includes a radiographic control apparatus 100, a radiographic apparatus 110, an X-ray interface 122, a radiation generation apparatus 120, an access point 130, an imaging apparatus 140, and radio wave reception apparatuses 150, which are connected via a network 160. The radiographic apparatus 110 is a portable flat panel detector (FPD). To obtain a radiographic image of a patient (subject) H lying on a bed 170, the radiographic apparatus 110 is inserted between the patient H and the bed 170. The patient H is then irradiated with radiation from an X-ray tube 123 based on an instruction from the radiation generation apparatus 120, and the radiographic apparatus 110 obtains the radiographic image of the patient H. A cassette holder may be integrated within the bed 170 (under the patient H), and the radiographic apparatus 110 may be inserted into the cassette holder.

The radiographic system is connected by the network 160 and a backbone network. The network 160 may be a wired or wireless network.

The radiographic control apparatus 100 is an apparatus that communicates with the radiographic apparatus 110 via the access point 130 to control radiographic imaging and obtain the radiographic image of the patient H obtained by the radiographic apparatus 110. The radiographic control apparatus 100 includes an information processing apparatus such as a computer (not shown).

The radiographic control apparatus 100 also communicates with the radiation generation apparatus 120 via the X-ray interface 122 to control radiographic imaging.

The radiographic control apparatus 100 also communicates with the imaging apparatus 140 to control the imaging apparatus 140 and obtain an image captured by the imaging apparatus 140.

The radiographic control apparatus 100 also communicates with the radio wave reception apparatuses 150 to obtain radio wave information that the radio wave reception apparatuses 150 has obtained by performing short-distance wireless communication with the radiographic apparatus 110.

The radiographic apparatus 110, which is an apparatus that transitions to an imaging state based on an instruction from the radiographic control apparatus 100, performs radiographic imaging in synchronization with the radiation generation apparatus 120, and generates a radiographic image based on the radiation emitted from the radiation generation apparatus 120. The number of radiographic apparatuses 110 is not limited to one, and the radiographic system may be configured to use a plurality of radiographic apparatuses 110. In the present exemplary embodiment, the radiographic apparatus 110 performs wireless communication using an Institute of Electrical and Electronics Engineers (IEEE) 802.11 standard with the radiographic control apparatus 100 via the access point 130. However, the radiographic apparatus 110 may be configured to connect to the network 160 in a wired manner.

The radiographic apparatus 110 has radio wave transmission apparatuses 111 attached outside, and performs short-distance wireless communication with reception units 151 of the radio wave reception apparatuses 150. The configuration of each of the radio wave transmission apparatuses 111 and the radio wave reception apparatuses 150 is not limited as long as a communication method corresponding to the short-distance wireless communication is implemented. For example, in some embodiments, the radio wave transmission apparatuses 111 and the radio wave reception apparatuses 150 may use short-range wireless technology standards, such as Bluetooth® or Ultra-wideband (UWB). In other embodiments, the radio wave transmission apparatuses 111 and the radio wave reception apparatuses 150 may use near field communication (NFC) standards, such as radio-frequency identification (RFID).

In the present exemplary embodiment, the radiographic apparatus 110 has a substantially rectangular shape. Radio wave transmission apparatuses 111A, 111B, 111C, and 111D are attached to the four corners of the radiographic apparatus 110. However, the configuration of the radio wave transmission apparatuses 111 is not limited to the above example as long as at least one radio wave transmission unit is disposed on a surface of the radiographic apparatus 110. FIG. 1 shows four radio wave transmission apparatuses 111 disposed at the four corners of the radiographic apparatus 110. In this example, the radio wave transmission apparatuses 111 may be built in the radiographic apparatus 110, or fixedly attached to a grid mounted on the radiographic apparatus 110. However, in some embodiments, the radio wave transmission apparatuses 111 can be configured to be detachable since the detachable radio wave transmission apparatuses 111 on the radiographic apparatus 110 can be used interchangeably in different radiographic apparatuses to implement the present exemplary embodiment.

The radiation generation apparatus 120 is an apparatus that detects an operator's radiation operation instruction issued from an exposure switch 121 and generates radiation using the tube 123 based on an irradiation condition set from a user input device (not illustrated) such as an operation panel.

The radiation generation apparatus 120 controls execution of the radiation operation instruction based on a synchronization instruction given by the radiographic control apparatus 100 via the X-ray interface 122. The X-ray interface 122 is an example of a control unit configured to control the radiation generation apparatus 120.

The access point 130 is an apparatus that wirelessly communicates with the radiographic apparatus 110 using the IEEE 802.11 standard, and communicates with the radiographic control apparatus 100 in a wired manner via the network 160.

The imaging apparatus 140 is an apparatus that performs imaging to obtain a captured image based on an instruction from the radiographic control apparatus 100. In the present exemplary embodiment, an optical camera is used as the imaging apparatus 140, and obtains an optical image as the captured image. However, the configuration of the imaging apparatus 140 is not limited as long as captured image information can be obtained. In the present exemplary embodiment, the imaging apparatus 140 is attached to the tube 123, and captures an image in the direction of radiation emission by the tube 123.

The radio wave reception apparatuses 150 include the reception units 151, and perform short-distance wireless communication with the radio wave transmission apparatuses 111 on the radiographic apparatus 110. In the present exemplary embodiment, three radio wave reception apparatuses 150A, 150B, and 150C and three reception units 151A, 151B, and 151C are used at positions where radio waves from the radio wave transmission apparatuses 111 can be received. However, the number and position of radio wave reception apparatuses is not limited as long as radio waves from the radio wave transmission apparatuses 111 can be received. For example, the radiographic control apparatus 100 may be configured to also serve as one of the radio wave reception apparatuses 150. One of the reception units 151 of the radio wave reception apparatuses 150 is attached to and disposed at the same position as the imaging apparatus 140. In the present exemplary embodiment, the reception unit 151A of the radio wave reception apparatus 150A is so attached and disposed.

The radio wave reception apparatuses 150 and the reception units 151 may be integrated with each other. In such a case, one of the radio wave reception apparatuses 150 is attached to and disposed at the same position as the imaging apparatus 140.

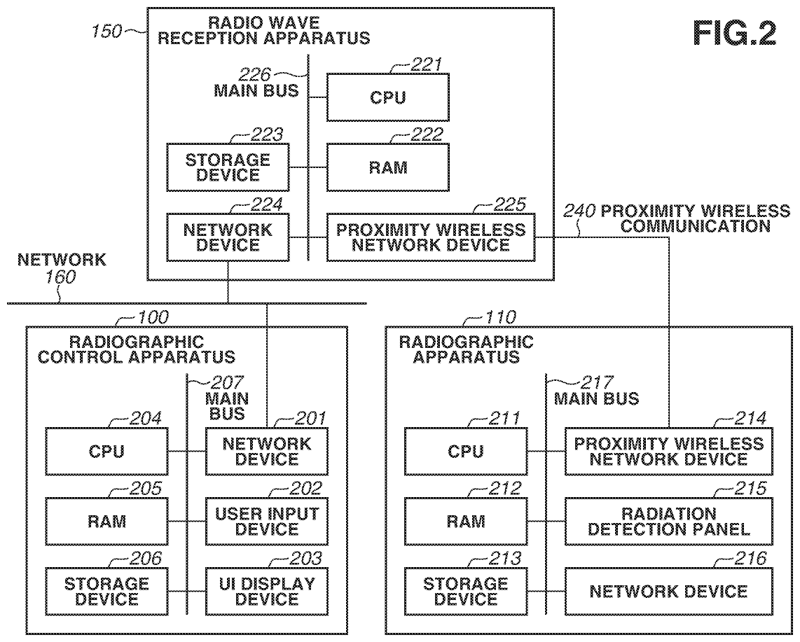

FIG. 2 illustrates a hardware configuration example of the radiographic system including the radiographic control apparatus 100, the radiographic apparatus 110, and a radio wave reception apparatus 150 according to the present exemplary embodiment.

The radiographic control apparatus 100 includes a network device 201 that connects to the network 160, a user input device 202 such as a keyboard that accepts user operations, and a user interface (UI) display device 203 such as a liquid crystal display that displays an operation screen and a radiographic image.

The radiographic control apparatus 100 further includes a central processing unit (CPU) 204 that controls the entire radiographic control apparatus 100, a random access memory (RAM) 205 that provides a work area for the CPU 204, and a storage device 206 that stores various control programs and radiographic images received from the radiographic apparatus 110.

The devices constituting the radiographic control apparatus 100 are connected by a main bus 207 and can transmit and receive data to and from each other.

While the user input device 202 and the UI display device 203 are described as separate devices, the user input device 202 and the UI display device 203 may be integrated into an operation unit.

The radiographic apparatus 110 includes a CPU 211 that controls the entire radiographic apparatus 110, a RAM 212 that provides a work area for the CPU 211, and a storage device 213 that stores various control programs and generated radiographic images.

The radiographic apparatus 110 further includes a proximity wireless network device 214 that communicates with the radio wave reception apparatus 150 using proximity wireless communication 240 compliant with a communication protocol for short-distance wireless communication connection. The proximity wireless network device 214 is provided for the radiographic apparatus 110 as a part of a hardware configuration of the radio wave transmission apparatuses 111.

The radiographic apparatus 110 further includes a radiation detection panel 215. The radiation detection panel 215 includes a flat panel detector (FPD), for example, and generates a radiographic image by generating electrical signals based on an amount of radiation.

The radiographic apparatus 110 further includes a network device 216 that communicates with the network 160 using wireless communication complaint with a communication protocol for wireless communication connection via the access point 130.

The devices constituting the radiographic apparatus 110 are connected by a main bus 217 and can transmit and receive data to and from each other.

The radio wave reception apparatus 150 includes a CPU 221 that controls the entire radio wave reception apparatus 150, a RAM 222 that provides a work area for the CPU 221, a storage device 223 that stores various control programs, and a network device 224 for connecting to the network 160.

The radio wave reception apparatus 150 further includes a proximity wireless network device 225 that communicates with the radiographic apparatus 110 using the proximity wireless communication 240 complaint with the communication protocol for short-distance wireless communication connection. The proximity wireless network device 225 is provided for the radio wave reception apparatus 150 as a part of the hardware configuration of the reception unit 151.

The devices constituting the radio wave reception apparatus 150 are connected by a main bus 226 and can transmit and receive data to and from each other.

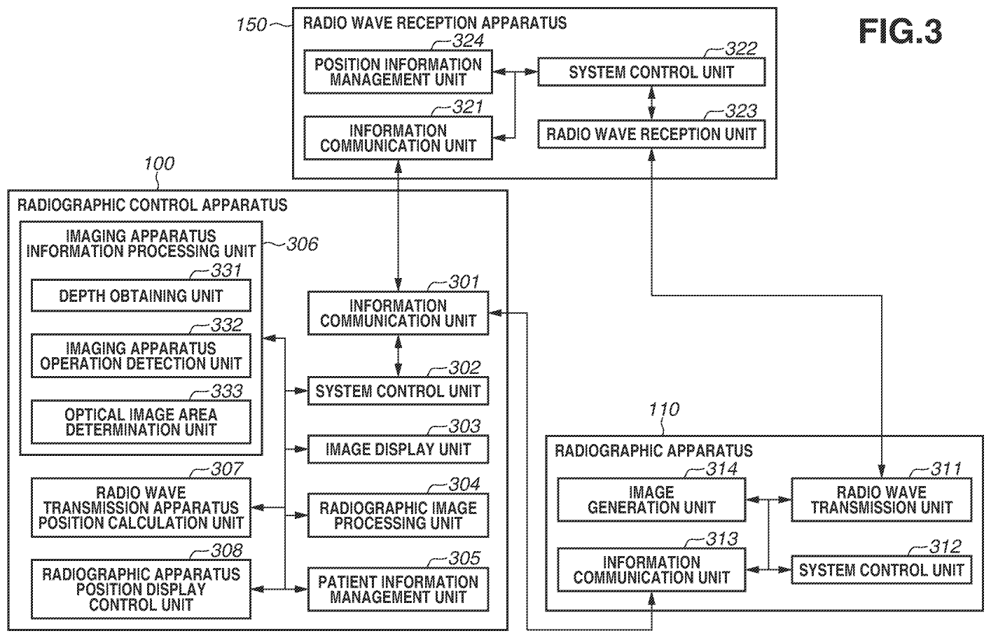

FIG. 3 illustrates a software configuration example of the radiographic system including the radiographic control apparatus 100, the radiographic apparatus 110, and the radio wave reception apparatus 150 according to the present exemplary embodiment.

The functional units illustrated in FIG. 3 are implemented by the CPUs 204, 211, and 221 of the respective apparatuses reading the control programs in the storage devices 206, 213, and 223 into the RAMs 205, 212, and 222, and executing the control programs.

The radiographic control apparatus 100 includes an information communication unit 301, a system control unit 302, an image display unit 303, a radiographic image processing unit 304, a patient information management unit 305, an imaging apparatus information processing unit 306, a radio wave transmission apparatus position calculation unit 307, and a radiographic apparatus position display control unit 308.

The information communication unit 301 is a software module for controlling the network device 201 to perform communication.

The system control unit 302 performs irradiation control on the radiation generation apparatus 120 and the radiographic apparatus 110, manages various states of the radiation generation apparatus 120 and the radiographic apparatus 110, and transmits and receives various types of setting information including setting information about the access point 130, via the information communication unit 301.

The system control unit 302 obtains a captured radiographic image from the radiographic apparatus 110 via the information communication unit 301.

The system control unit 302 further obtains an optical image from the imaging apparatus 140 and radio wave information about the radio wave transmission apparatuses 111 and position information about the reception units 151 from the radio wave reception apparatuses 150, via the information communication unit 301.

The system control unit 302 is a program for implementing basic functions of the radiographic control apparatus 100, and controls operation of various units.

The image display unit 303 displays images and information generated by the radiographic image processing unit 304, the imaging apparatus information processing unit 306, and the radiographic apparatus position display control unit 308 via the UI display device 203.

Moreover, the image display unit 303 reflects image processing instructed by the system control unit 302 and performs processing for switching a screen display of the UI display device 203 based on operations from the user input device 202. In other words, the image display unit 303 corresponds to an example of a display control unit configured to display an optical image on a display unit.

The radiographic image processing unit 304 processes the captured radiographic image obtained via the system control unit 302 to generate an image to be used on the radiographic control apparatus 100.

The patient information management unit 305 manages an imaging order received from a Radiology Information System (RIS) server (not illustrated) via the network 160, and controls the radiographic apparatus 110 and the radiation generation apparatus 120 based on the imaging order.

The patient information management unit 305 also manages the captured radiographic image generated by the radiographic image processing unit 304 and irradiation information (such as a tube voltage and a tube current) about the radiation generation apparatus 120 in association with the imaging order, and transmits the resultant to an image server (not illustrated) via the network 160. With the management and the transmission processing by the patient information management unit 305, the image and information generated by the imaging apparatus information processing unit 306 and the radiographic apparatus position display control unit 308 may also be associated.

The imaging apparatus information processing unit 306 processes the optical image obtained via the system control unit 302 to generate an image and information to be used on the radiographic control apparatus 100. The imaging apparatus information processing unit 306 includes a depth obtaining unit 331, an imaging apparatus operation detection unit 332, and an optical image area determination unit 333.

The depth obtaining unit 331 processes an optical image and generates depth information about one or more objects shown in the optical image. In other words, the depth obtaining unit 331 corresponds to a software module configured to obtain depth information in an optical image from the optical image. In the present exemplary embodiment, unsupervised learning for monocular depth estimation using a convolutional neural network (CNN)-based machine learning algorithm is used for the processing of the depth obtaining unit. The depth obtaining unit 331 has a trained data set (not illustrated) including images captured in advance and distance information, and makes an inference from an optical image. More specifically, the depth obtaining unit 331 can obtain depth information about an area to which the radiographic apparatus 110 belongs in an optical image (information about a distance between the imaging apparatus 140 and the radiographic apparatus 110) by inputting an optical image captured by the imaging apparatus 140 to the machine learning algorithm that is trained to receive input of an optical image and output depth information. However, the configuration is not limited as long as a method for generating depth information in an optical image is implemented. For example, a time-of-flight camera, a radar sensor, or an optical camera with an ultrasonic sensor (not illustrated) may be used as the imaging apparatus 140 to directly obtain depth data from the sensor. A stereo camera (not illustrated) may be used as the imaging apparatus 140 to estimate depth data based on an optical image obtained as a stereoscopic image.

The imaging apparatus operation detection unit 332 detects operation information (rotation and translation) about the imaging apparatus 140 attached to the tube 123. In other words, the imaging apparatus operation detection unit 332 corresponds to an example of a detection unit configured to detect operation information about the imaging apparatus 140. In the present exemplary embodiment, for the processing, relative operation information about the imaging apparatus 140 is inferred from a positional relationship between the points of view of an object in a reference, previous, and subsequent optical images among optical images successively obtained by the imaging apparatus 140, using a CNN-based machine learning algorithm. More specifically, the imaging apparatus operation detection unit 332 can detect the operation information about the imaging apparatus 140 by inputting the optical images captured by the imaging apparatus 140 to a machine learning algorithm that is trained to receive input of optical images and output operation information. However, the configuration is not limited as long as a method for obtaining the operation information about the imaging apparatus 140 is implemented. For example, the imaging apparatus 140 may include various sensors (not illustrated) such as an acceleration sensor, a gyro sensor, and a geomagnetic sensor, and obtain relative operation information from the various sensors.

The optical image area determination unit 333 processes an optical image and generates area division information in the optical image. In other words, the optical image area determination unit 333 corresponds to an example of an identification unit configured to identify an object area in an optical image. In the present exemplary embodiment, area extraction based on a machine learning algorithm such as U-Net is used for the processing. The optical image area determination unit 333 has a trained data set (not illustrated) for identifying the patient H, the radiographic apparatus 110, the bed 170, and a background area, and makes an inference from the optical image. More specifically, the optical image area determination unit 333 can identify the area to which the radiographic apparatus 110 belongs in the optical image captured by the imaging apparatus 140 by inputting the optical image captured by the imaging apparatus 140 to a machine learning algorithm that is trained to receive input of an optical image and identify areas in the optical image. The optical image area determination unit 333 can further identify the subject, the bed 170, and the background area. However, the configuration is not limited as long as a method capable of identifying the patient H, the radiographic apparatus 110, the bed 170, and the background area in the optical image is implemented. The optical image area determination unit 333 may be implemented by a combination of known image area division algorithms instead of the machine learning algorithm.

To accurately calculate the position information about the radio wave transmission apparatuses 111 even in a case where the radiographic apparatus 110 is hidden behind the patient H, the optical image area determination unit 333 is desirably capable of identifying alternative area information (about the subject, the bed 170, and the background area) for calculating the position information about the radiographic apparatus 110. However, the optical image area determination unit 333 may at least be capable of identifying the area to which the radiographic apparatus 110 belongs.

The radio wave transmission apparatus position calculation unit 307 calculates the distances between the radio wave transmission apparatuses 111 and the reception units 151 and the position information about the radio wave transmission apparatuses 111 based on the radio wave information about the radio wave transmission apparatuses 111 and the position information about the reception units 151 obtained via the system control unit 302. In other words, the radio wave transmission apparatus position calculation unit 307 corresponds to an example of a calculation unit that calculates the distance between a transmission unit, which is configured to transmit radio waves and disposed on the radiographic apparatus configured to generate a radiographic image of a subject based on radiation emitted from the radiation generation apparatus, and a reception unit, which is configured to receive the radio waves, and position information about the transmission unit based on the radio field intensity of the transmission unit and the position information about the reception unit.

The radio wave transmission apparatus position calculation unit 307 corrects the radio wave information to be used in calculating the distances between the radio wave transmission apparatuses 111 and the reception units 151, and the position information about the radio wave transmission apparatuses 111, based on depth information in the optical image obtained via the radiographic apparatus position display control unit 308. In other words, the radio wave transmission apparatus position calculation unit 307 corresponds to an example of a correction unit configured to correct the position information about the transmission unit calculated by the calculation unit using depth information about the area to which the radiographic apparatus belongs in the optical image captured by the imaging apparatus 140, the depth information being obtained from the optical image.

The radiographic apparatus position display control unit 308 calculates on-screen position information about the radio wave transmission apparatuses 111. Here, the radiographic apparatus position display control unit 308 uses the distances between the radio wave transmission apparatuses 111 and the reception units 151 and the position information about the radio wave transmission apparatuses 111 obtained via the radio wave transmission apparatus position calculation unit 307 and the operation information about the imaging apparatus 140 obtained via the imaging apparatus information processing unit 306. Then, the radiographic apparatus position display control unit 308 generates an area image of the radiographic apparatus 110 to be used on the radiographic control apparatus 100.

Moreover, the radiographic apparatus position display control unit 308 notifies the radio wave transmission apparatus position calculation unit 307 of the operation information about the imaging apparatus 140 and the depth information in the optical image, obtained via the imaging apparatus information processing unit 306.

The radiographic apparatus 110 includes a radio wave transmission unit 311, a system control unit 312, an information communication unit 313, and an image generation unit 314.

The radio wave transmission unit 311 is a software module for controlling the proximity wireless network device 214 to perform communication.

The system control unit 312 transmits and receives irradiation control information and state management information about the radiographic apparatus 110 to and from the radiographic control apparatus 100 via the information communication unit 313.

The system control unit 312 also transmits an image generated by the image generation unit 314 to the radiographic control apparatus 100 via the information communication unit 313.

The system control unit 312 further transmits and receives various types of setting information including wireless information to and from the access point 130 via the information communication unit 313. The system control unit 312 further transmits radio wave information to the radio wave reception apparatus 150 via the radio wave transmission unit 311.

The system control unit 312 is a program for implementing basic functions of the radiographic apparatus 110, and controls operation of various components.

The information communication unit 313 is a software module for controlling the network device 216 to perform communication.

The image generation unit 314 obtains a radiographic image resulting from radiation irradiation, and corrects the radiographic image using a dark image obtained without radiation irradiation to generate a captured radiographic image.

The radio wave reception apparatus 150 includes an information communication unit 321, a system control unit 322, a radio wave reception unit 323, and a position information management unit 324.

The information communication unit 321 is a software module for controlling the network device 224 to perform communication.

The system control unit 322 transmits the radio wave information received from the radio wave transmission apparatuses 111 of the radiographic apparatus 110 and position information about the system control unit 322 obtained from the position information management unit 324 to the radiographic control apparatus 100 via the information communication unit 321.

The system control unit 322 receives the radio wave information from the radio wave transmission apparatuses 111 of the radiographic apparatus 110 via the radio wave reception unit 323.

The radio wave reception unit 323 is a software module for controlling the proximity wireless network device 225 to perform communication.

The position information management unit 324 manages information about the current position of the reception unit 151 of the radio wave reception apparatus 150. The current position here may be any information from which the position with respect to the imaging apparatus 140 can be identified. For example, relative position information with respect to a reference position of the imaging apparatus 140 may be measured in advance, and the measured relative position information may be set in the position information management unit 324. A Global Positioning System (GPS) receiver (not illustrated) for receiving signals from GPS satellites may be included to calculate the position information.

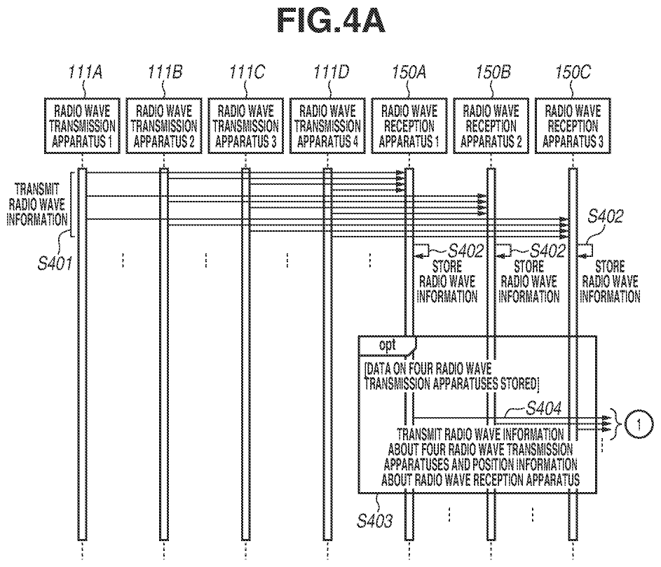

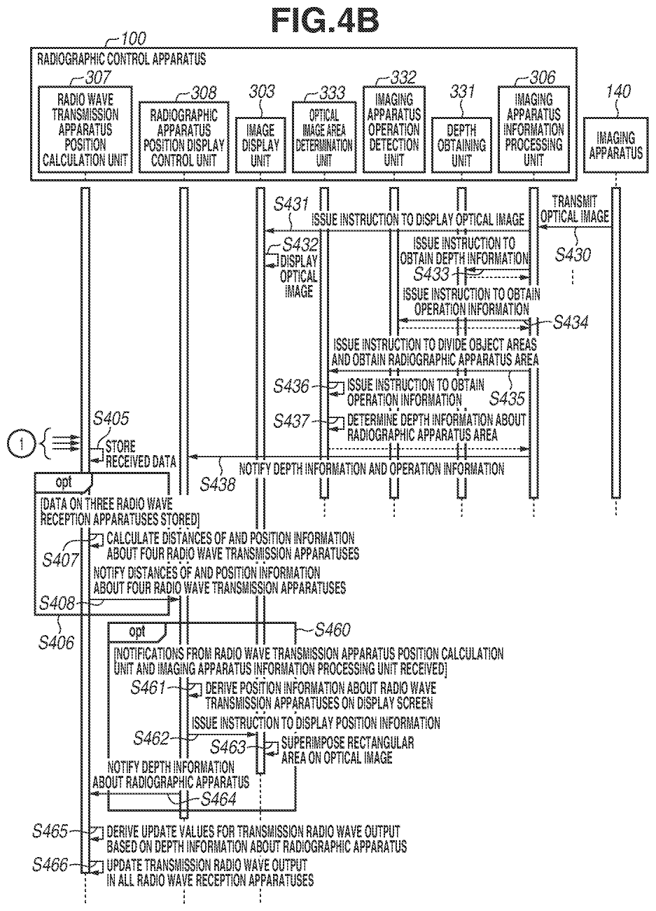

FIGS. 4A and 4B illustrate a method for superimposition processing of the area image of the radiographic apparatus 110 and the correction processing of the radio wave information using the radiographic control apparatus 100, the radio wave transmission apparatuses 111, the radio wave reception apparatuses 150, and the imaging apparatus 140 according to the present exemplary embodiment.

In step S401, the radio wave transmission apparatuses 111 transmit radio wave information to radio wave reception units 323 of the radio wave reception apparatuses 150 via radio wave transmission units 311. The radio wave transmission apparatuses 111 transmit the radio wave information a plurality of times at constant intervals.

In step S402, the radio wave reception apparatuses 150 store the radio wave information received from the radio wave transmission apparatuses 111. The radio wave information to be stored includes the received signal strength indication (RSSI) and transmission radio wave output power (TxPower) of each of the radio wave transmission apparatuses 111A to 111D. To reduce the effect of fluctuations in the RSSI radio field intensity, the radio wave reception apparatuses 150 may be configured to store a corrected RSSI, for example, by determining the quartiles of the last 10 RSSIs from the target radio wave transmission apparatus 111 and storing an average of the first to third quartiles. TxPowers are values set by the radio wave transmission apparatuses 111 in advance. TxPowers may therefore be obtained only the first time, or preset in the radio wave reception apparatuses 150.

In step S403, the radio wave reception apparatuses 150 determine whether the RSSIs and TxPowers of the respective radio wave transmission apparatuses 111A to 111D are stored. If the RSSIs and TxPowers are stored, then in step S404, the radio wave reception apparatuses 150 transmit the radio wave information (RSSI and TxPower) about each of the radio wave transmission apparatuses 111 to the radio wave transmission apparatus position calculation unit 307 of the radiographic control apparatus 100 via the information communication units 321. Here, the radio wave reception apparatuses 150 also transmit the position information thereof stored in their position information management units 324. The radio wave reception apparatuses 150 perform the transmission processing of step S404 each time the radio wave information is received in step S402 and the affirmative determination is made in step S403.

In step S405, the radio wave transmission apparatus position calculation unit 307 stores the information received from the radio wave reception apparatuses 150. The received information to be stored includes the position information about each of the radio wave reception apparatuses 150A to 150C, and the radio wave information (RSSI and TxPower) about each of the radio wave transmission apparatuses 111 notified from each of the radio wave reception apparatuses 150.

In step S406, the radio wave transmission apparatus position calculation unit 307 determines whether the received information about each of the radio wave reception apparatuses 150A to 150C is stored. If the received information is stored, then in step S407, the radio wave transmission apparatus position calculation unit 307 calculates the distances of and the position information about the respective radio wave transmission apparatuses 111A to 111D.

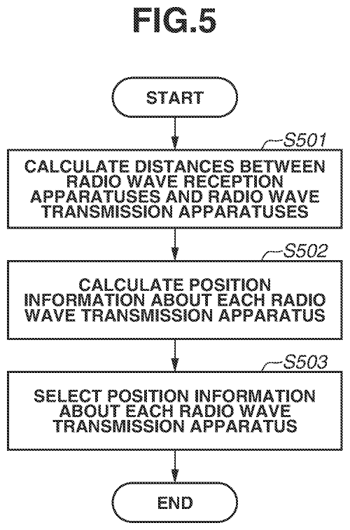

The distance and position information derivation processing by the radio wave transmission apparatus position calculation unit 307 in step S407 will now be described with reference to FIG. 5.

In step S501, the radio wave transmission apparatus position calculation unit 307 calculates the distances between the radio wave reception apparatuses 150 and the radio wave transmission apparatuses 111. A distance d_{bjpi} between each radio wave transmission apparatus 111bⱼ (j=A, B, C, and D) and each radio wave reception apparatus 150pᵢ (i=A, B, and C) is determined based on the Friis transmission equation using the following Eq. (1).

d_{b}_{ⱼ}ₚ_{ᵢ}=10{(TxPower_{b}_{ⱼ}−RSSI_{b}_{ⱼ}ₚ_{ᵢ})/10N} (1)

Here, TxPower_{bj} is the TxPower of each radio wave transmission apparatus 111bⱼ, and RSSI_{bjpi} is the RSSI of each radio wave transmission apparatus 111bⱼ received by each radio wave reception apparatus 150p₁. N is a propagation loss factor indicating the quality of the radio wave transmission path, and set depending on the environment.

In step S502, the radio wave transmission apparatus position calculation unit 307 calculates the position information about each radio wave transmission apparatus 111bⱼ. Here, the radio wave transmission apparatus position calculation unit 307 determines intersections of spheres having radii d_{bjpi} about the positions (x_{piw}, y_{piw}, z_{piw}) of the respective radio wave reception apparatuses 150pᵢ. The position (x_{bjw}, y_{bjw}, z_{bjw}) of each radio wave transmission apparatus 111bⱼ is derived by deriving the intersections of three spherical surfaces.

$$\{ \begin{matrix}\begin{matrix}{( {x}_{{p}_{A} w} - {x}_{{b}_{j} w} )}^{2} + {( {y}_{{p}_{A} w} - {y}_{{b}_{j} w} )}^{2} + {( {z}_{{p}_{A} w} - {z}_{{b}_{j} w} )}^{2} = {d}_{{b}_{j} {p}_{A}}^{2} \\ {( {x}_{{p}_{B} w} - {x}_{{b}_{j} w} )}^{2} + {( {y}_{{p}_{B} w} - {y}_{{b}_{j} w} )}^{2} + {( {z}_{{p}_{B} w} - {z}_{{b}_{j} w} )}^{2} = {d}_{{b}_{j} {p}_{B}}^{2}\end{matrix} \\ {( {x}_{{p}_{C} w} - {x}_{{b}_{j} w} )}^{2} + {( {y}_{{p}_{C} w} - {y}_{{b}_{j} w} )}^{2} + {( {z}_{{p}_{C} w} - {z}_{{b}_{j} w} )}^{2} = {d}_{{b}_{j} {p}_{C}}^{2}\end{matrix} \qquad ( 2 )$$

There can be two intersections of the three spherical surfaces or no intersection as a result of calculation. In step S503, the radio wave transmission apparatus position calculation unit 307 therefore selects position information about each radio wave transmission apparatus 111bⱼ. The radio wave transmission apparatus position calculation unit 307 solves Exp. (3) that minimizes errors in the positions (x_{piw}, y_{piw}, z_{piw}) of the radio wave reception apparatuses 150p1 and the distances d_{bjpi}.

$${min}_{{b}_{j}} \frac{1}{M} {\sum }_{i = 1}^{M} \{ \sqrt{{( {x}_{{p}_{i} w} - {x}_{{b}_{j} w} )}^{2} + {( {y}_{{p}_{i} w} - {y}_{{b}_{j} w} )}^{2} + {( {z}_{iw} - {z}_{{b}_{j} w} )}^{2}} - {d}_{{b}_{j} {p}_{i}} \} \qquad ( 3 )$$

The radio wave transmission apparatus position calculation unit 307 derives the positions (x_{bjw}, y_{bjw}, z_{bjw}) of the radio wave transmission apparatus 111bⱼ from the calculations. Here, M is the total number of radio wave reception apparatuses 150. In the present exemplary embodiment, M is three.

The radio wave transmission apparatus position calculation unit 307 employs an intersection with the smaller error in the distances to the point derived by Exp. (3) between the two intersections derived in step S502. If no intersection is successfully derived in step S502, a point derived using Exp. (3) is employed as the intersection.

Return to the description of the processing of FIGS. 4A and 4B.

In step S408, the radio wave transmission apparatus position calculation unit 307 notifies the radiographic apparatus position display control unit 308 of the distances of and the position information about the respective radio wave transmission apparatuses 111A to 111D derived in step S407.

Processing related to the imaging apparatus 140 will now be described.

In step S430, the imaging apparatus 140 successively transmits captured optical images to the imaging apparatus information processing unit 306 via the information communication unit 301 of the radiographic control apparatus 100.

The processing is started by the imaging apparatus information processing unit 306 issuing an imaging start instruction (not illustrated) to the imaging apparatus 140 in advance. The processing starts with a notification from the system control unit 302 about transition of the radiographic control apparatus 100 to an inspection start state based on a user operation (not illustrated).

In step S431, the imaging apparatus information processing unit 306 instructs the image display unit 303 to display an optical image transmitted in step S430. Receiving the instruction of step S431, then in step S432, the image display unit 303 performs processing for displaying the optical image.

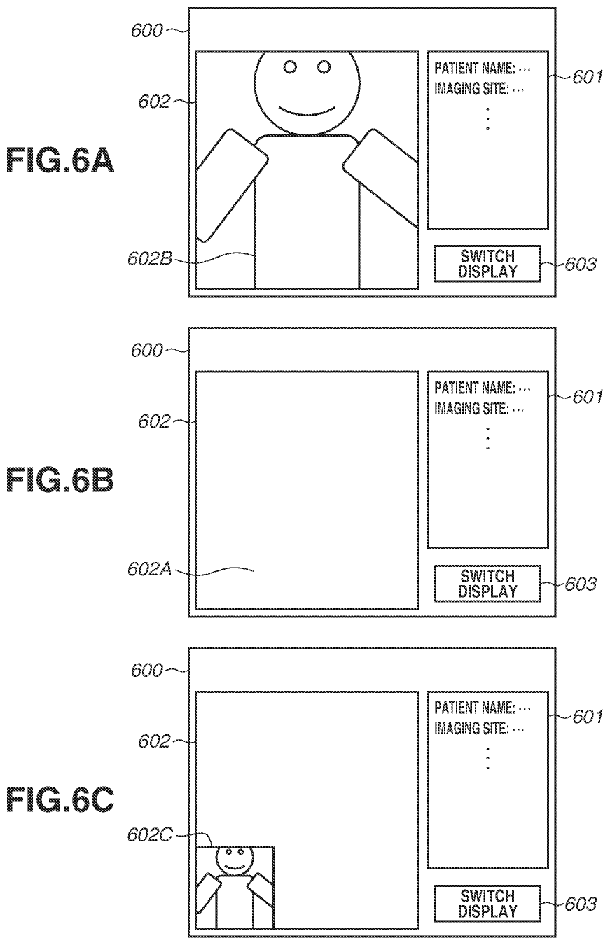

A screen display by the image display unit 303 will be described with reference to FIGS. 6A to 6C. An inspection screen 600 is a screen displayed when the radiographic control apparatus 100 transitions to the inspection start state based on a user operation. The user checks and inputs inspection information and performs zoom in and out processing and gradation processing on a radiographic image via an operation section 601.

The inspection screen 600 also includes a screen display area 602 via which a radiographic image and an optical image are displayed, and a display switch section 603 that is used to switch the screen display area 602 from a screen display area 602A to a screen display area 602C. The screen display area 602A shows an example of the screen display area 602 in a radiographic image display state. At the time of step S432, no image is displayed since no radiographic image is received from the radiographic apparatus 110. A screen display area 602B shows an example of the screen display area 602 in an optical image display state. The screen display area 602C shows an example of the screen display area 602 in a state of superimposition of an optical image on the display of a radiographic image. If the radiographic control apparatus 100 includes a plurality of UI display devices 203, the screen display area 602 of the inspection screen 600 may be extended to display a radiographic image and an optical image separately.

In step S433, the imaging apparatus information processing unit 306 instructs the depth obtaining unit 331 to obtain depth information, and obtains depth information about the entire optical image. In the present exemplary embodiment, the depth obtaining unit 331 inputs the optical image and obtains depth information about the entire optical image.

In step S434, the imaging apparatus information processing unit 306 instructs the imaging apparatus operation detection unit 332 to obtain operation information, and obtains operation information about the imaging apparatus 140. In the present exemplary embodiment, the imaging apparatus operation detection unit 332 inputs the optical image and obtains relative rotation and translation information about the imaging apparatus 140 accompanying the tube 123.

In step S435, the imaging apparatus information processing unit 306 instructs the optical image area determination unit 333 to divide object areas and obtain the area to which the radiographic apparatus 110 belongs (hereinafter, referred to as a radiographic apparatus 110 area), and obtains depth information about the radiographic apparatus 110 area. In the present exemplary embodiment, the optical image area determination unit 333 inputs the optical image and the depth information about the entire optical image, and obtains the depth information about the radiographic apparatus 110 area.

In step S436, the optical image area determination unit 333 divides the input optical image into areas and obtains area division information about the optical image. In step S437, the optical image area determination unit 333 determines the depth information about the radiographic apparatus 110 area based on the result of step S436 and the depth information about the entire input optical image.

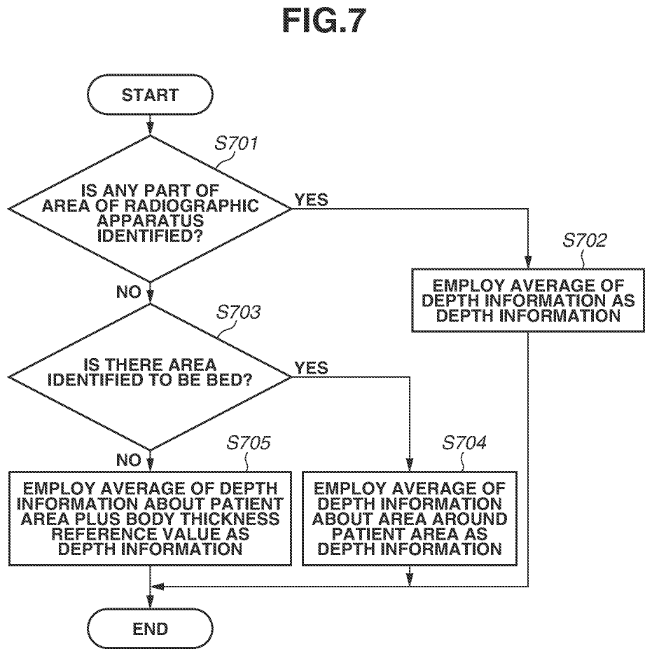

The depth information determination processing of the radiographic apparatus 110 area by the optical image area determination unit 333 in step S437 will now be described with reference to FIG. 7. When the radiographic apparatus 110 is hidden behind the patient H and is difficult to be identified from the optical image, depth information about an approximate area is employed by the determination processing of FIG. 7.

In step S701, the optical image area determination unit 333 determines whether there is any part of the area of the radiographic apparatus 110 identified in step S436. If, in step S701, the optical image area determination unit 333 determines that there is such an area (YES in step S701), the processing proceeds to step S702. In step S702, the optical image area determination unit 333 calculates an average of the depth information about the area, and employs the calculated average as the depth information about the radiographic apparatus 110. In other words, the position information about the radio wave transmission apparatuses 111 can be corrected using the depth information about the identified area to which the radiographic apparatus 110 belongs in the optical image.

If, in step S701, the optical image area determination unit 333 determines that there is no such area (NO in step S701), the processing proceeds to step S703. In step S703, the optical image area determination unit 333 determines whether there is an area identified to be an area of the bed 170 behind the patient H. If, in step S703, the optical image area determination unit 333 determines that there is such an area (YES in step S703), the processing proceeds to step S704. In step S704, the optical image area determination unit 333 calculates an average of the depth information about an area in the vicinity of a patient area in the area, and employs the calculation result as the depth information about the radiographic apparatus 110. The vicinity of the patient area is defined as an area around and slightly larger than the area determined to be the patient area by the area division, or defined by a bounding box surrounding the patient area. A depth value obtained as the calculation result may be subjected to processing for subtracting a numerical value corresponding to previously measured thickness information about the radiographic apparatus 110 from the depth value. If the cassette holder under the bed 170 is used, the depth value may be subjected to processing for adding a numerical value corresponding to distance information about the cassette holder to the depth value.

If, in step S703, the optical image area determination unit 333 determines that there is no such area (NO in step S703), the processing proceeds to step S705. In step S705, the optical image area determination unit 333 calculates an average of the depth information about the patient area, adds a body thickness reference value set in advance to the calculated average, and employs the sum as the depth information about the radiographic apparatus 110. Examples of the body thickness reference value may include a body thickness value used for a dose index provided as a diagnostic reference level for medical exposure, and a reference value set by each medical institution measuring patients' body thicknesses. A body thickness value derived from the height and weight based on physical size information about the patient H may be used. In other words, the optical image area determination unit 333 can obtain depth information about each area identified in the optical image.

Returning now to the description of the processing of FIGS. 4A and 4B.

In step S438, the imaging apparatus information processing unit 306 notifies the radiographic apparatus position display control unit 308 of the depth information about the radiographic apparatus 110 area obtained in step S433 and the operation information about the imaging apparatus 140 obtained in step S434.

Processing related to the radiographic apparatus position display control unit 308 will be described.

In step S460, the radiographic apparatus position display control unit 308 determines whether the notification of the distances and the position information from the radio wave transmission apparatus position calculation unit 307 and the notification of the depth information and the operation information from the imaging apparatus information processing unit 306 have been received. If the notifications are determined to have been received, then in step S461, the radiographic apparatus position display control unit 308 derives position information about the radio wave transmission apparatuses 111 on the display screen.

In step S461, the radiographic apparatus position display control unit 308 uses a projection model for converting the positions (x_{bjw}, y_{bjw}, z_{bjw}) of the radio wave transmission apparatuses 111bⱼ in the three-dimensional space captured by the imaging apparatus 140 into positions (ubⱼ, vbⱼ) on a two-dimensional image, which is given by Eq. (4):

$$[ \begin{matrix}{u}_{{b}_{j}} \\ {v}_{{b}_{j}} \\ 1\end{matrix} ] = A [ R \vert t ] [ \begin{matrix}{x}_{{b}_{j} w} \\ {y}_{{b}_{j} w} \\ {z}_{{b}_{j} w} \\ 1\end{matrix} ] \qquad ( 4 )$$

Here, A is an internal parameter of the imaging apparatus 140 and has been obtained by calibration of the imaging apparatus 140 in advance. [R|t] is an external parameter of the imaging apparatus 140 and expresses rotation and translation. The operation information about the imaging apparatus 140 obtained in step S438 is used for [R|t].

In step S462, the radiographic apparatus position display control unit 308 controls the image display unit 303 to display the position information about the respective radio wave transmission apparatuses 111 on the display screen, obtained in step S461. In step S463, the image display unit 303, in response to the instruction given in step S462, generates a rectangular area representing the radiographic apparatus 110 by connecting points with lines based on the position information, and performs superimposition processing on the optical image. In other words, the image display unit 303 can further display area information about the radiographic apparatus 110 estimated based on the position information about the transmission units disposed on the radiographic apparatus 110 in the optical image. The image display unit 303 can display the area formed by connecting, with lines, the positions of the plurality of transmission units disposed on the radiographic apparatus 110 in the optical image as the area information about the radiographic apparatus 110.

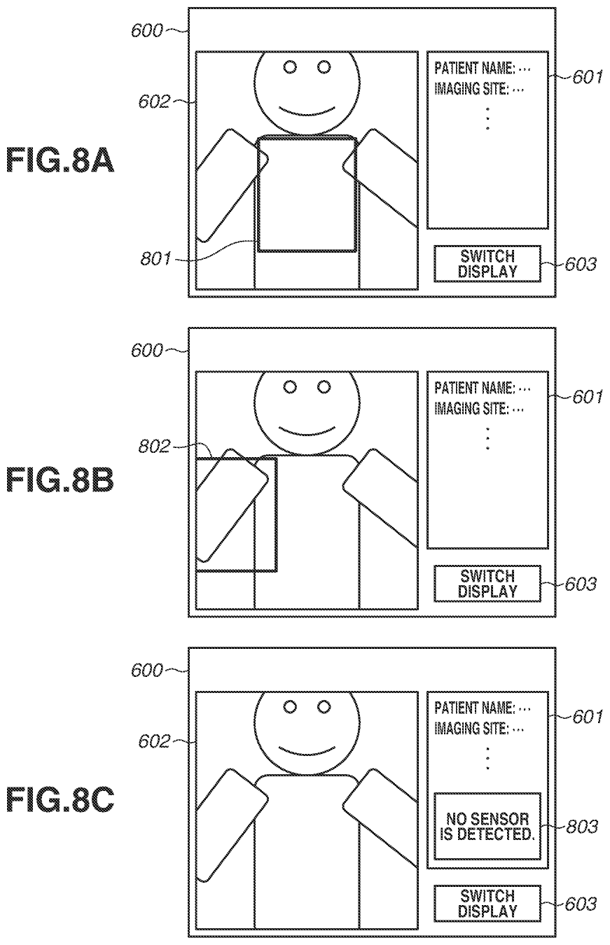

The screen display of the image display unit 303 will be described with reference to FIGS. 8A to 8C illustrating differences from FIGS. 6A to 6C. Estimated radiographic apparatus position area information 801 cornered with the radio wave transmission apparatuses 111 is successively superimposed on the optical image in the screen display area 602. If some of the radio wave transmission apparatuses 111 are located outside the field of view of the optical image, the rectangular area lying within the field of view of the optical image is successively superimposed on the optical image as in estimated radiographic apparatus position area information 802. In step S460, in a case where the notification of the distances and the position information from the radio wave transmission apparatus position calculation unit 307 (step S408) is unable to be obtained or in a case where all the radio wave transmission apparatuses 111 are located outside the field of view of the imaging apparatus 140 (i.e., not seen in the optical image), a message 803 (see FIG. 8C) indicating such a case is displayed on-screen. Here, the screen display area 602 does not display the estimated radiographic apparatus position area information 802. The message 803 is an example of an error message in the case where the notification of the distances and the position information is unable to be obtained in step S460. In the case where all the radio wave transmission apparatuses 111 are located outside the field of view of the optical image, a message indicating that the radiographic apparatus 110 is outside the field of view of the optical image, such as “the sensor position is outside the screen”, is displayed.

In step S464, the radiographic apparatus position display control unit 308 notifies the radio wave transmission apparatus position calculation unit 307 of the depth information about the radiographic apparatus 110 from the imaging apparatus information processing unit 306. The notification processing may be performed only in a case where there is a large error between the distances notified from the radio wave transmission apparatus position calculation unit 307 and the depth information notified from the imaging apparatus information processing unit 306. Specifically, the radiographic apparatus position display control unit 308 may calculate errors between the distances of the respective radio wave transmission apparatuses 111 from the radio wave reception apparatus 150A and the depth information about the radiographic apparatus 110. If any of the distance errors is greater than or equal to a threshold, the radiographic apparatus position display control unit 308 may notify the radio wave transmission apparatus position calculation unit 307 of the depth information about the radiographic apparatus 110 along with the information about the radio wave transmission apparatus(es) 111 in question.

In step S465, the radio wave transmission apparatus position calculation unit 307 derives update values for the transmission radio wave output (TxPower) of the radio wave transmission apparatuses 111 based on the depth information about the radiographic apparatus 110. In other words, the radio wave transmission apparatus position calculation unit 307 corrects the position information about the radio wave transmission apparatuses 111 by correcting the radio field intensities based on the depth information about the area to which the radiographic apparatus 110 belongs. In the processing of this step, the radio wave transmission apparatus position calculation unit 307 derives update values TxPower′_{bj} with respect to the radio wave reception apparatus 150A (150p_{A}) attached at the same position as the imaging apparatus 140 based on the RSSIs of the respective radio wave transmission apparatuses 111bⱼ and depth information d_{depth} about the radiographic apparatus 110, using transformed Eq. (1) that is Eq. (5):

TxPower′_{bj}=RSSI_{bjPA}+10N log₁₀d_{depth} (5)

In step S466, the radio wave transmission apparatus position calculation unit 307 instructs all the radio wave reception apparatuses 150 to update the transmission radio wave output of the respective radio wave transmission apparatuses 111 with the update values TxPower′_{bj} calculated in step S465.

Note that the information to be updated in steps S465 and S466 may be the propagation loss factor N.

More specifically, Eq. (5) in step S465 may be transformed to derive the propagation loss factor N. In step S466, the radio wave transmission apparatus position calculation unit 307 may instruct all the radio wave reception apparatuses 150 to update the propagation loss factors N of the respective radio wave transmission apparatuses 111 with the derived values.

Step S466 enables the use of updated TxPower in calculating the distances of the respective radio wave transmission apparatuses 111 for use in the series of processes of position estimation processing of the radiographic apparatus 110 in FIGS. 4A and 4B.

In such a manner, a series of processes of the processing by the radiographic system according to the present exemplary embodiment is performed.

As described above, the radio field intensities depending on radio wave attenuation due to the radiographic apparatus 110 being hidden behind the patient H, in which case the depth information about the radiographic apparatus 110 is likely to deviate from the distances between the radio wave reception apparatuses 150 and the radio wave transmission apparatuses 111, can be corrected. In other words, the estimation accuracy of the positions of the radio wave transmission apparatuses 111 can be improved. The detection accuracy of the position information about the radiographic apparatus 110 displayed on the screen of the radiographic system can thus be improved.

Moreover, to address the issue of the radio wave attenuation in the case where the radiographic apparatus 110 is hidden behind the patient H, the setting values of the radio field intensities used in deriving distance information, based on which the position information about the radiographic apparatus 110 is calculated, can be corrected with the depth information obtained from the optical image. In other words, the effect of the radio wave attenuation can be reduced to improve the detection accuracy of the position information about the radiographic apparatus 110 displayed on the screen of the radiographic system.

Next, a second exemplary embodiment of the present disclosure will be described.

In the configuration according to the first exemplary embodiment, the transmission radio wave output of the radio wave transmission apparatuses 111 for use in the position estimation processing of the radiographic apparatus 110 is updated using the depth information about the radiographic apparatus 110 to improve the detection accuracy. However, depending on selection of the imaging apparatus 140, in situations where the accurate depth estimation is difficult, for example, due to imaging in a dark place, the accuracy can rather be decreased by the update.

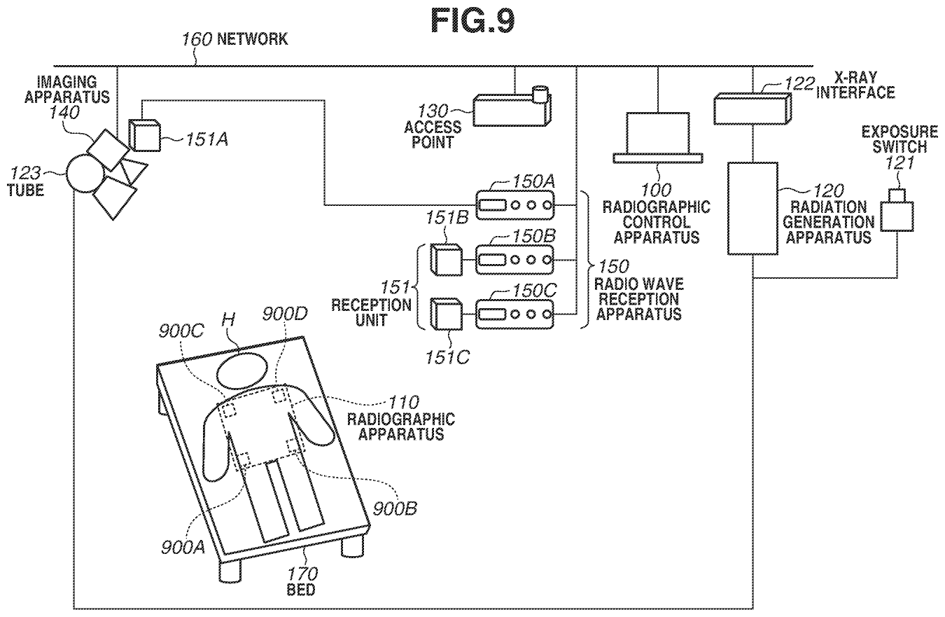

In the configuration according to the second exemplary embodiment, there is added processing for correcting the depth information about the radiographic apparatus 110 obtained from the imaging apparatus 140 using the distances between the radio wave reception apparatuses 150 and radio wave transmission apparatuses 900. Only differences from the first exemplary embodiment will hereinafter be described with reference to FIGS. 9 to 12.

FIG. 9 illustrates a configuration example of the entire radiographic system according to the present exemplary embodiment.

The radio wave transmission apparatuses 900 according to the present exemplary embodiment have a marker for identifying each of the radio wave transmission apparatuses 900 on a surface thereof. The marker may use any identification information, such as barcode, shape, and color information, as long as the radio wave transmission apparatuses 900 can be identified on an optical image obtained by the imaging apparatus 140.

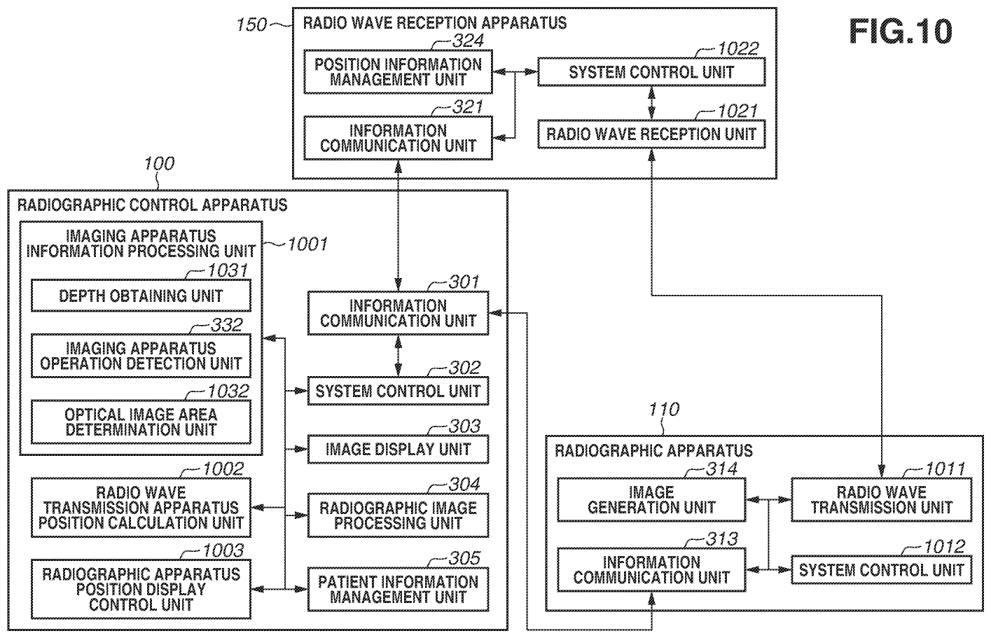

FIG. 10 illustrates a software configuration example of the radiographic system including the radiographic control apparatus 100, the radiographic apparatus 110, and a radio wave reception apparatus 150 according to the present exemplary embodiment.

An imaging apparatus information processing unit 1001 according to the present exemplary embodiment includes a depth obtaining unit 1031 and an optical image area determination unit 1032 in addition to the functions of the imaging apparatus information processing unit 306.

The depth obtaining unit 1031 has a function of correcting a depth estimation result based on depth correction values notified from a radiographic apparatus position display control unit 1003 in addition to the functions of the depth obtaining unit 331.

The optical image area determination unit 1032 has a function of identifying the radio wave transmission apparatuses 900 in the optical image in addition to the functions of the optical image area determination unit 333. The configuration of the identification processing is not limited, and a known image processing algorithm such as a pattern recognition algorithm may be used for implementation. Moreover, the imaging apparatus information processing unit 1001 generates identification identifiers (IDs) of the radio wave transmission apparatuses 900 identified by the optical image area determination unit 1032 in the optical image and additional depth information related to the identified areas.

A radio wave transmission apparatus position calculation unit 1002 according to the present exemplary embodiment has a function of processing information about the radio wave transmission apparatuses 900 along with the identification IDs in addition to the functions of the radio wave transmission apparatus position calculation unit 307.

The radiographic apparatus position display control unit 1003 according to the present exemplary embodiment has a function of receiving the identification IDs of the radio wave transmission apparatuses 900 in the optical image and the depth information related to the identified areas from the imaging apparatus information processing unit 1001 in addition to the functions of the radiographic apparatus position display control unit 308. When the depth information is obtained, the radiographic apparatus position display control unit 1003 calculates depth correction values based on the distances between the corresponding radio wave transmission apparatuses 900 and the reception units 151, and notifies the imaging apparatus information processing unit 1001 of the depth correction values.

In other words, the position information about the radio wave transmission apparatuses 900 is corrected using the depth information about the radio wave transmission apparatuses 900 identified in the optical image based on the identification information.

A system control unit 1012 according to the present exemplary embodiment has a function of adding the identification IDs, such as unique serial IDs, of the radio wave transmission apparatuses 900 to transmission radio waves as additional radio wave information in transmitting the radio wave information to the radio wave reception apparatuses 150 in addition to the functions of the system control unit 312. A radio wave transmission unit 1011 communicates and transmits the added radio wave information.

A radio wave reception unit 1021 according to the present exemplary embodiment receives radio waves to which the identification IDs, such as unique serial IDs, of the radio wave transmission apparatuses 900 are added as radio wave information during communication. A system control unit 1022 has a function of obtaining the identification IDs from the radio wave information and transmitting the radio wave information and the identification IDs to the radiographic control apparatus 100 in addition to the functions of the system control unit 322. The radio wave reception unit 1021, similar to the radio wave reception unit 323, is software that controls the proximity wireless network device 225 to perform communication.

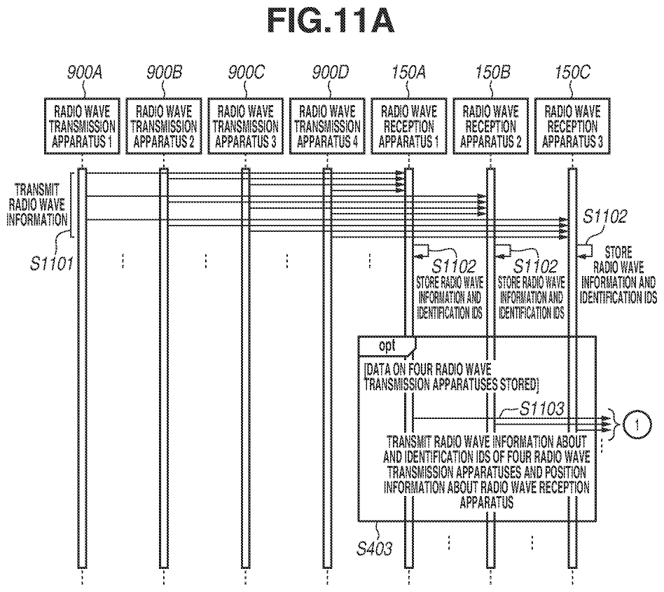

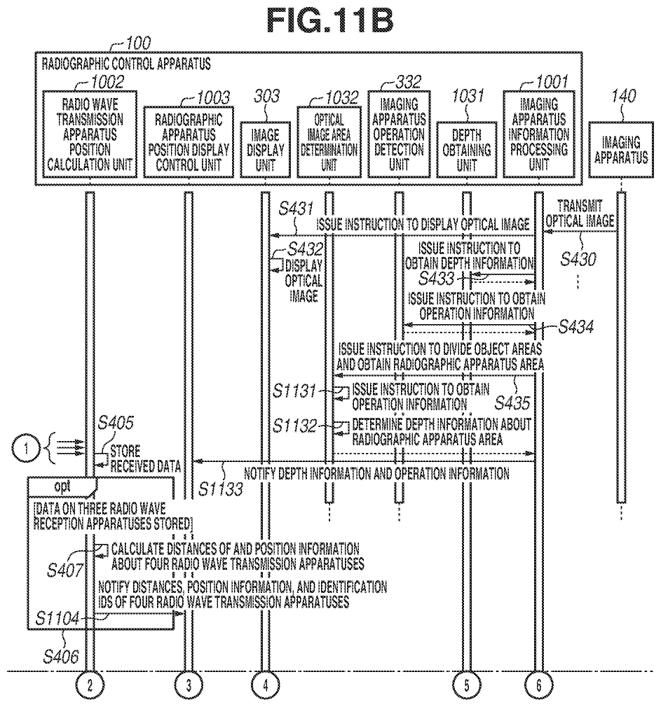

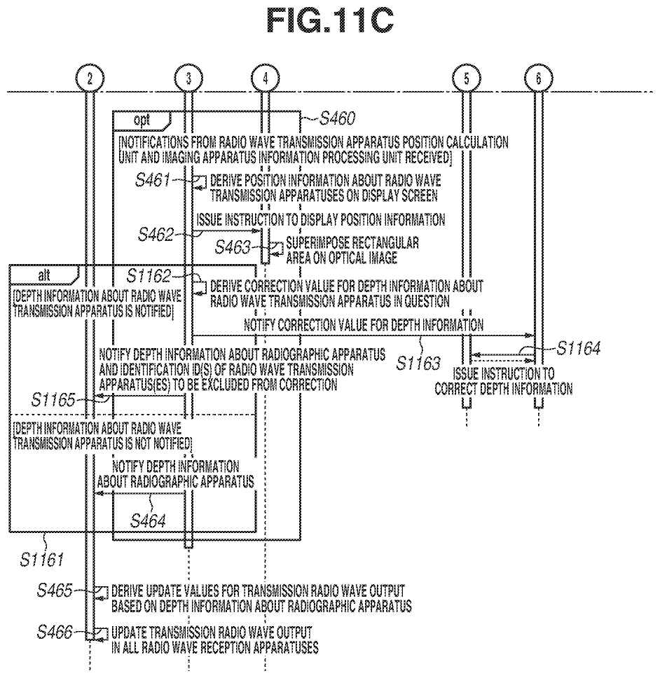

FIGS. 11A, 11B, and 11C illustrate a method for the superimposition processing of the area image of the radiographic apparatus 110 and the correction processing of the radio wave information and the depth information using the radiographic control apparatus 100, the radio wave transmission apparatuses 900, the radio wave reception apparatuses 150, and the imaging apparatus 140 according to the present exemplary embodiment.

In step S1101, the radio wave transmission apparatuses 900 transmit radio wave information to radio wave reception units 1021 of the radio wave reception apparatuses 150 via radio wave transmission units 1011. The identification IDs of the radio wave transmission apparatuses 900 are added to the radio wave information. The radio wave transmission apparatuses 900 transmit the radio wave information a plurality of times at constant intervals.

In step S1102, the radio wave reception apparatuses 150 store the radio wave information received from the radio wave transmission apparatuses 900. The radio wave information to be stored includes the received signal strength indication RSSI, the transmission radio wave output (TxPower), and the identification ID of each of the radio wave transmission apparatuses 900A to 900D. To reduce the effect of fluctuations in the RSSI radio field intensity, the radio wave reception apparatuses 150 may be configured to store a corrected RSSI, for example, by determining the quartiles of the last 10 RSSIs from the target radio wave transmission apparatus 900 and storing an average of the first to third quartiles. TxPowers are values set by the radio wave transmission apparatuses 900 in advance. TxPowers may therefore be obtained only the first time, or preset in the radio wave reception apparatuses 150. Similarly, the identification IDs also remain unchanged during the radio wave reception processing, and may therefore be obtained only the first time.

In step S1103, the radio wave reception apparatuses 150 transmit the radio wave information (RSSI, TxPower, and identification ID) about each of the radio wave transmission apparatuses 900 to the radio wave transmission apparatus position calculation unit 1002 of the radiographic control apparatus 100 via the information communication unit 321. Here, the radio wave reception apparatuses 150 also transmit the position information thereof stored in their position information management units 324. The radio wave reception apparatuses 150 perform the transmission processing of step S1103 each time the radio wave information is received in step S1102 and the affirmative determination is made in step S403.

In step S1104, the radio wave transmission apparatus position calculation unit 1002 notifies the radiographic apparatus position display control unit 1003 of the distances, the position information, and the identification IDs of the respective radio wave transmission apparatuses 111A to 111D derived in step S407.

In step S1131, the optical image area determination unit 1032 divides the input optical image into areas and obtains area division information about the optical image. In addition, the optical image area determination unit 1032 identifies the areas of the radio wave transmission apparatuses 900 in the optical image. In step S1132, the optical image area determination unit 1032 determines the depth information about the radiographic apparatus 110 area based on the result of step S1131 and the depth information about the entire input optical image.

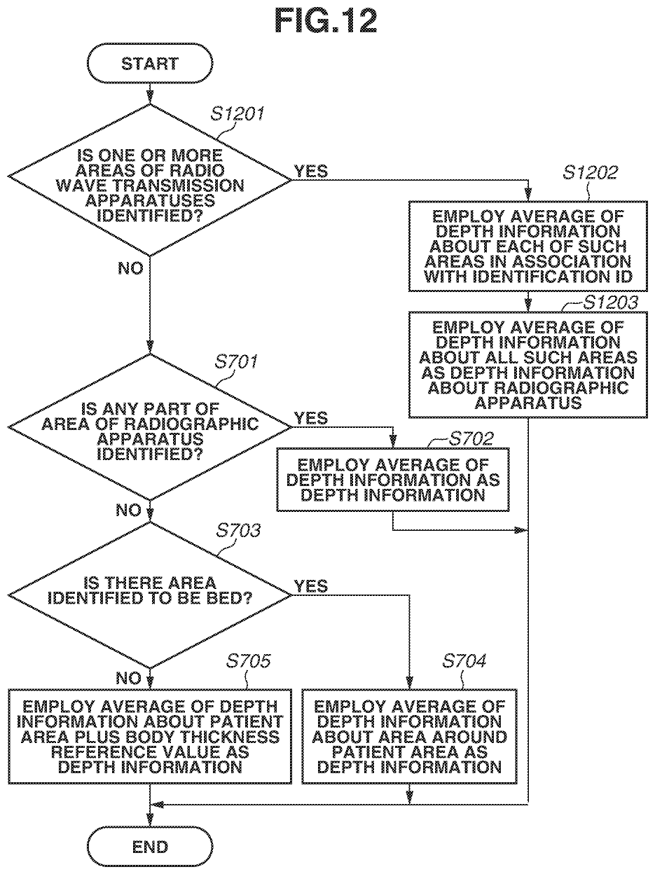

The depth information determination processing of the radiographic apparatus 110 area by the optical image area determination unit 1032 in step S1132 will now be described with reference to FIG. 12. The determination processing according to the present exemplary embodiment includes employing the depth information about an approximate area through the determination processing illustrated in FIG. 7, and if the areas of the radio wave transmission apparatuses 900 in the optical image are successfully identified, additionally obtaining depth information about the identified areas.

In step S1201, the optical image area determination unit 1032 determines whether there is one or more areas of the radio wave transmission apparatuses 900 identified in step S1131. If, in step S1201, the optical image area determination unit 1032 determines that there is such an area or areas (YES in step S1201), the processing proceeds to step S1202. In step S1202, the optical image area determination unit 1032 calculates an average of the depth information about each of such areas of the radio wave transmission apparatuses 900, and employs the calculated average in association with the identification ID of the radio wave transmission apparatus 900. In step S1203, the optical image area determination unit 1032 also calculates an average of the depth information about all such areas, and employs the calculated average as the depth information about the radiographic apparatus 110.

If, in step S1201, the optical image area determination unit 1032 determines that there is no such area (NO in step S1201), the processing proceeds to step S701 as in FIG. 7.

In step S1133, the imaging apparatus information processing unit 1001 notifies the radiographic apparatus position display control unit 1003 of the depth information about the radiographic apparatus 110 area and the operation information about the imaging apparatus 140 obtained before step S1133. In step S1133, the imaging apparatus information processing unit 1001 also notifies the radiographic apparatus position display control unit 1003 of the identification IDs of and the depth information about the radio wave transmission apparatuses 900 obtained in step S1132. The imaging apparatus information processing unit 1001 obtains such information attached to the results output in response to the instructions of step S435 (not illustrated).

In step S1161, the radiographic apparatus position display control unit 1003 determines whether the identification ID of and the depth information about any of the radio wave transmission apparatuses 900 are included in the depth information obtained from the imaging apparatus information processing unit 1001. If the identification ID and the depth information are determined to be included, then in step S1162, the radiographic apparatus position display control unit 1003 obtains distance errors between the distances from the respective radio wave reception apparatuses 150 to the radio wave transmission apparatus 900 having the identification ID and the depth information, and derives a correction value. The processing of this step is performed for the distance d_{bjpA} of the radio wave transmission apparatus 900bⱼ having the identification ID to the radio wave reception apparatus 150A (150p_{A}) disposed at the same position as the imaging apparatus 140. If there is more than one radio wave transmission apparatus 900 in question, the correction value is derived by obtaining an average value or employing a correction value with a distance error greater than or equal to a threshold.

In step S1163, the radiographic apparatus position display control unit 1003 notifies the imaging apparatus information processing unit 1001 of the correction value for the depth information derived in step S1162.

In step S1164, the imaging apparatus information processing unit 1001 instructs the depth obtaining unit 1031 to correct the depth information. In the present exemplary embodiment, the depth obtaining unit 1031 inputs the correction value for the depth information and adds the correction value to the depth values of an entire optical image derived from a processing result by the depth obtaining unit 1031.

In step S1165, the radiographic apparatus position display control unit 1003 notifies the radio wave transmission apparatus position calculation unit 1002 of the depth information about the radiographic apparatus 110 from the imaging apparatus information processing unit 1001 and the identification ID(s) of the radio wave transmission apparatus(es) 900 to be excluded from the notified correction. The processing of step S465 and the subsequent step, which is similar to the processing thereof in FIGS. 4A and 4B, is performed only on the radio wave transmission apparatus(s) 900bⱼ having no identification ID.

If, in step S1161, no such identification ID or depth information is determined to be included, the processing proceeds to step S464 that is similar to the processing thereof in FIGS. 4A and 4B.

By the foregoing processing, the depth obtaining unit 1031 can perform distance estimation while correcting the depth information about the radiographic apparatus 110 based on the distances between the radio wave reception apparatuses 150 and the radio wave transmission apparatuses 900. In other words, in situations where the accurate depth estimation using the imaging apparatus 140 is difficult, the effect of the update processing decreasing the accuracy can be reduced.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the claims are not limited to the disclosed exemplary embodiments. The scope of the following claims is to be accorded the broadest interpretation so as to encompass all such modifications and equivalent structures and functions.

This application claims the benefit of Japanese Patent Application No. 2021-203549, filed Dec. 15, 2021, which is hereby incorporated by reference herein in its entirety.

What is claimed is:

1. A radiographic control apparatus comprising:
a position information obtaining unit configured to obtain position information about a transmission unit configured to transmit a radio wave based on a radio field intensity of the transmission unit and position information about a reception unit configured to receive the radio wave, the transmission unit being disposed on a radiographic apparatus configured to generate a radiographic image of a subject based on radiation emitted from a radiation generation apparatus; and
a correction unit configured to correct the position information about the transmission unit using depth information about an area to which the radiographic apparatus belongs in an optical image captured by an imaging apparatus, the depth information being obtained from the optical image.

2. The radiographic control apparatus according to claim 1, further comprising a depth obtaining unit configured to obtain depth information in the optical image from the optical image,
wherein the correction unit is configured to correct the position information about the transmission unit using the depth information about the area to which the radiographic apparatus belongs, the depth information being obtained by the depth obtaining unit.

3. The radiographic control apparatus according to claim 2, wherein the depth obtaining unit is configured to input the optical image captured by the imaging apparatus to a machine learning algorithm and obtain the depth information about the area to which the radiographic apparatus belongs, the machine learning algorithm being trained to receive input of an optical image and output depth information.

4. The radiographic control apparatus according to claim 2, wherein the depth obtaining unit is configured to obtain the depth information using a stereo image obtained from a stereo camera used as the imaging apparatus, or depth data obtained from any one of the following used as the imaging apparatus: a time-of-flight camera, a radar sensor, and an optical camera with an ultrasonic sensor.

5. The radiographic control apparatus according to claim 2, further comprising an identification unit configured to identify an object area in the optical image,
wherein the position information about the transmission unit is corrected using the depth information about the area to which the radiographic apparatus belongs in the optical image, the area being identified by the identification unit.

6. The radiographic control apparatus according to claim 5, wherein the identification unit is configured to input the optical image captured by the imaging apparatus to a machine learning algorithm and identify the area to which the radiographic apparatus belongs in the optical image captured by the imaging apparatus, the machine learning algorithm being trained to receive input of an optical image and identify an area in the optical image.

7. The radiographic control apparatus according to claim 6, wherein the identification unit is configured to identify a subject, a radiographic apparatus, a bed, and a background area in the optical image.

8. The radiographic control apparatus according to claim 5, wherein the depth obtaining unit is configured to obtain depth information about each area identified by the identification unit in the optical image.

9. The radiographic control apparatus according to claim 1, wherein the correction unit is configured to correct the position information about the transmission unit by correcting the radio field intensity based on the depth information about the area to which the radiographic apparatus belongs.

10. The radiographic control apparatus according to claim 1, further comprising a detection unit configured to detect operation information about the imaging apparatus.

11. The radiographic control apparatus according to claim 10, wherein the detection unit is configured to input the optical image captured by the imaging apparatus to a machine learning algorithm and detect the operation information about the imaging apparatus, the machine learning algorithm being trained to receive input of an optical image and output operation information.

12. The radiographic control apparatus according to claim 1, further comprising a display control unit configured to display the optical image on a display unit,
wherein the display control unit is configured to further display area information about the radiographic apparatus estimated based on the position information about the transmission unit disposed on the radiographic apparatus in the optical image.

13. The radiographic control apparatus according to claim 12, wherein the display control unit is configured to display an area formed by connecting positions of a plurality of transmission units disposed on the radiographic apparatus in the optical image with lines as the area information about the radiographic apparatus.

14. The radiographic control apparatus according to claim 12, wherein the display control unit is configured to display the area information about the radiographic apparatus estimated based on the position information about the transmission unit, the position information being obtained based on operation information about the imaging apparatus.

15. A radiographic system comprising:
a radiographic apparatus configured to generate a radiographic image of a subject based on radiation emitted from a radiation generation apparatus, the radiographic apparatus including a transmission unit configured to transmit a radio wave;
a radiographic control apparatus configured to control imaging using the radiographic apparatus;
an imaging apparatus configured to capture an optical image; and
a reception apparatus configured to receive the radio wave transmitted from the transmission unit,
wherein the radiographic control apparatus includes
an obtaining unit configured to obtain position information about the transmission unit based on a radio field intensity of the transmission unit and position information about the reception apparatus, and
a correction unit configured to correct the position information about the transmission unit using depth information about an area to which the radiographic apparatus belongs in the optical image captured by the imaging apparatus, the depth information being obtained from the optical image.

16. The radiographic system according to claim 15,
wherein the radiographic apparatus is an apparatus of substantially rectangular shape,
wherein the transmission unit is disposed at each of four corners of the substantially rectangular shape, and
wherein at least three reception apparatuses are disposed at positions where radio waves from the transmission units are receivable.

17. The radiographic system according to claim 15,
wherein the transmission unit has identification information from which the transmission unit is identifiable in the optical image, and
wherein the correction unit is configured to correct the position information about the transmission unit using the depth information about the transmission unit identified in the optical image based on the identification information.

18. A radiographic control method comprising:
obtaining position information about a transmission unit configured to transmit a radio wave based on a radio field intensity of the transmission unit and position information about a reception unit configured to receive the radio wave, the transmission unit being disposed on a radiographic apparatus configured to generate a radiographic image of a subject based on radiation emitted from a radiation generation apparatus;
correcting the radio field intensity based on depth information about an area to which the radiographic apparatus belongs in an optical image, the optical image being captured in a direction of radiation emission of the radiation generation apparatus; and
obtaining position information about the transmission unit based on the corrected radio field intensity.

19. A non-transitory computer-readable storage medium storing a program for causing a computer to execute the method according to claim 18.
